(19) United States
(12) Patent Application Publication
(10) Pub. No.: US 2012/0208229 A1
(43) Pub. Date: Aug. 16, 2012

(54) DEVICE FOR EXAMINING CELLS HAVING AN ELASTOMER, AND USE OF THE DEVICE

(76) Inventors: Wolfgang Rubner, Herzogenrath (DE); Bernd Hoffmann, Juelich (DE); Sebastian Houben, Bonn-Bad Godesberg (DE); Rudolf Merkel, Juelich (DE)

(21) Appl. No.: 13/501,178

(22) PCT Filed: Sep. 25, 2010
(86) PCT No.: PCT/DE2010/001139

§ 371 Date: Apr. 12, 2012

(30) Foreign Application Priority Data
Oct. 14, 2009 (DE) 10 2009 049 454.4

(51) Int. Cl. C12M 1/34; C12Q 1/02

(52) U.S. Cl. 435/29; 435/287.1

(57) ABSTRACT

A device for examining cells comprising an elastomer and the use thereof. The elastomer comprises a bottom and a thicker edge region; and the bottom is provided with regular microstructures. Such elastomers are suitable for stretch experiments, notably of the uniaxial type.

The invention relates to a device for examining cells, comprising an elastomer, and to the use thereof.

In fundamental research and medical/clinical analysis, cell deformation systems make an important contribution to the simulation of cyclical deformations as they occur in animal tissue. These deformations, for example around arterial blood vessels or around the intestinal tract, constitute a signal for the cells and result in morphological and functional changes in the affected tissue, without which the functionality thereof would not be achieved.

Typically, cells are oriented at a defined angle with respect to an applied deformation. The angle may vary within a range of 60° and 90°, relative to the direction of deformation applied. Experimental simulation of the static or cyclic deformation of cells in a cell culture is carried out by means of so-called cell stretchers. Having knowledge of the reorientation of cells forms an important branch of research for the understanding of structured tissue.

Cell stretchers are divided into unidirectional, bidirectional, and equibiaxial stretchers, according to the experimental set-up. Equibiaxial cell stretchers generally utilize differing piston systems, over which an elastic membrane is stretched at one end. The membrane located on top is stretched by applying positive or negative pressure inside the piston by means of pneumatic actuation. Cells seeded on this membrane are thus exposed to deformation when they have adhered to the surface. A drawback of these systems is that isotropic stretching experiments supply results that are only useful to a very limited extent.

Uniaxial and biaxial cell stretchers employ electric motor drives. Typically, stepper motors or direct current motors are used. Elastic membranes, referred to as elastomers or chambers, which can be installed between a retaining device and a corresponding drive unit, can thus be uniaxially stretched. By using at least four, and typically eight, such drive units, it is also possible to sequentially and successively stretch an elastic membrane or chamber in two directions, in a biaxial manner.

The disadvantage of such biaxial cell stretchers according to the prior art is that, due to the number of motors and associated control electronics alone, they are expensive. Another disadvantage is that the cell stretchers according to the prior art do not allow the deformation of the elastomer and the cell forces to be determined, and the results that are determined cannot thus be used for simulating the response of cells, caused by stretching, in situ, which is to say in the tissue.

It is the object of the invention to develop a device for examining cells, comprising an elastomer, by means of which the influence of external mechanical stress on the cells can be better examined.

The object of the invention is achieved by the device according to claim 1 and the use thereof according to the additional independent claim. Advantageous embodiments will be apparent from the respective dependent claims.

The elastomer made of cross-linked silicone oil (polydimethylsiloxane, PDMS) is designed as a chamber, which also serves as a cell culture vessel during stretching experiments. The elastomer chamber is used to accommodate cells. This means that cells are seeded on the elastomer and adhere to the surface. For this purpose, the elastomer has a bottom and a thicker edge region.

The bottom is provided with regular microstructures, to which the cells adhere. The diameters of the microstructures and the distances between the microstructures in the bottom are advantageously in the micrometer range, and typically range between 1 and 10 μm. The depth of the microstructure can be adjusted and varied so as to be completely unrecognized by the cells, or so as to be deliberately recognized. In the experiments, the microstructure serves as a gauge, which allows the stretching applied to the elastomer and the forces acting on the cells to be determined.

The devices according to the invention, for the first time, allow the difference between the stretch that is applied to the elastomer chamber and the stretch that is actually received at the cells to be determined. The stretch of the chamber in the X-direction brings about a transverse contraction, and thus a compression of the elastomer, in the Y-direction. This results in a cushion-shaped deformation of the elastomer chamber, and notably of the chamber bottom, to which the cells adhere. The progression of the deformation of the chamber bottom can be exactly determined by identifying the microstructure. In addition, when the modulus of elasticity of the elastomer is known, it is also possible, for the first time, to determine the forces of adhering cells in cyclical stretch experiments.

The identification of the microstructure is possible both for calibration purposes, using no seeded cells, and while carrying out an experiment over time. It is thus possible, for the first time, to provide exact forecasts as to the anticipated orientation angle of the cells and to set experimental parameters so that these are optimally suitable for checking the predictions.

It was found, within the context of the invention, that a device for examining the cells can be used to precisely determine the influence of a static or cyclic force action on the cells, using an elastomer. To this end, cells are placed on an elastomer, or in general terms on an elastic substrate and, after the cells have adhered thereto, the substrate is statically or cyclically strained, which is to say the cells are deformed together with the bottom. It was found that the reorientation of the cells depends on the transverse contraction of the substrate that is used, which is to say by the extent to which the cells are compressed during cyclic stretch experiments. Poisson's ratio describes the ratio of contraction (in the Y-direction) to stretch (in the X-direction) of an elastic substrate, wherein the change in thickness of the chamber bottom (in the Z-direction) can be neglected because the adhering cells only experience the deformation field in the X-Y plane. Poisson's ratio is material-specific, for example, being 0.5 when using PDMS as the substrate material. The compression of the chamber and the bottom can be varied by corresponding geometric shaping and stiffening.

It was found, within the context of the invention, that the devices of the prior art cannot be used to precisely characterize the stretch applied to the cell, the determination of the forces, and the determination of the transverse contraction. It was further found that the transverse contraction of the elastomer chamber decisively influences the behavior of the cell, notably in terms of reorientation. Because the cell chambers in cell stretchers known from the prior art are subject to imprecision in this regard, large errors occur in evaluation of the experiments.

The elastic substrate preferably has a rectangular, and for example quadratic, tub shape, because the negative molds for this purpose are simple and cost-effective to produce, notably with respect to the production of chambers using, for example, a casting method.

The elastomer particularly advantageously comprises openings in the edge region. The openings are hole-like and can penetrate, preferably in the Z-direction, the entire edge of the elastomer. The openings constitute guides, by which the elastomer is anchored to the motor drive of the cell stretcher in a stable manner.

The openings are particularly advantageously automatically introduced in the edge, during the production process of the elastomer chamber using a common cross-linking process. For this purpose, pins having the dimensions desired for the openings are introduced in the negative mold at the corresponding locations of the negative mold. The pins have the same dimensions as the fastening pins in the cell stretcher for the elastomer. The polymer (PDMS silicone oil) is mixed with a copolymer (cross-linking agent), and the elastomer, while still liquid, is poured into the negative mold and, while still in liquid form in the non-cross-linked state, encloses the pins. Thereafter the PDMS is cured or cross-linked according to the manufacturer's instructions. Particularly advantageously, the openings in the elastomer are form fitted with the mounting of the motor for the cell stretcher after curing, so as to optimally and stably connect the elastomer to the fastenings of the cell stretcher.

The elastomer preferably comprises an edge region having rounded corners. Advantageously, and as differs from the sharp-edge transitions according to the prior art, these do not tear, even under uniform loads resulting from frequent cyclical stretch and compression.

The rounded corners can preferably be present in the edge region, oriented outwardly, and/or in the edge region, oriented inwardly. Because the openings are arranged in the round corners, and the elastomer is thereby connected to the retaining pins of the cell stretcher, particularly good force transmission to the chamber bottom, and hence to the cells, by the cell stretcher, is achieved when the substrate is stretched. Because of the more favorable force progression that results, the rounded corners transmit the applied amplitude of the stretcher considerably more exactly than the angular corners known from the prior art.

In a further embodiment of the invention, the wall thickness of the corners of the elastomer chamber is greater than the wall thickness in the remaining edge region. This advantageously results in further stabilization of the chamber during stretch experiments.

The elastomer particularly advantageously has a shape in which the wall thicknesses of the opposing edge regions are identical, and in which the non-opposing edge regions can be equally as thick or have a different thickness. The wall height is constant. The elastomer chamber can particularly advantageously be varied so as to adjust the transverse contraction of the chamber bottom by way of the wall thickness of the edge of the elastomer chamber parallel to the direction of tension. Because of this, cells can reorient at the same stretching amplitude.

Examination using low compression and large stretch (small transverse contraction of the substrate) is made possible by producing the edge of the elastomer that is parallel to the direction of tension so as to be less resilient than the remaining edge regions.

However, the edge region of the elastomer that is parallel to the direction of tension can also be interrupted or left entirely open. This will increase the transverse contraction. Maximum transverse contraction is reached when the bottom of the elastomer chamber is freely suspended, which is to say, that the elastomer chamber has no edge region parallel in the direction of tension. It is thus possible to simulate biaxial cell stretch systems using uniaxial cell stretch systems, by adjusting the transverse contraction, and to study the corresponding cell behavior. Because known biaxial cell stretch systems cost several times more than uniaxial systems, this is a major step toward cost reduction.

The edge region of the elastomer perpendicular to the direction of tension can particularly advantageously be reinforced. The reinforcement brings about particularly uniform force transmission to the elastomer bottom, and thus to the cells seeded thereon. The reinforcement may also comprise clamp-shaped materials having a positive fit with respect to the elastomer, such as angle brackets. These additional mountings, which are perpendicular to the direction of tension, prevent the edge region from deflecting in the direction of tension and, when the chamber is inserted in the cell stretcher, considerably improve the ratio of the applied chamber stretch, and the stretch that is actually received by the cells. The use of additional mountings or angle brackets also causes a more uniform stretch progression in the chamber bottom, and thus a considerably larger region in which the behavior of the cells can be examined.

In addition to determining the transverse contraction, the microstructure can also be used for analyzing the cell force of cells before, during and after the substrate stretch. This allows, for the first time, the simultaneous multidimensional data analysis of two physical parameters (tensile and cell forces). The resolution limit of cell force analyses under tension can also be increased in the chambers that are used, by introducing fluorescent nanospheres in the chamber bottom.

In principle, the microstructure can be generated by means of stamps, such as those described in German patent application DE 10 2005 005 121. However, the invention is not limited to these. The structure can rather also be generated by means of fluorescent nanospheres and similar regular structures.

Elastomer chambers can also be produced in varying elasticities, or lined with a thin layer of elastomers having varying elasticities. The device advantageously comprises an elastomer having an elasticity in the range of 0.1 kPa to 1 MPa.

The drive system that is used is preferably a commercial linear drive, with which the user can freely set all the parameters, such as the speed and travel range, as part of the technical specifications of the selected drive.

Additional freely selectable parameters include the holding times of the starting and end positions, and optionally individual or cyclical stretch. To this end, the number of cycles, or optionally the duration of the experiment, can be freely selected.

The drive is self-calibrating and has an automatic zero position relative to the geometry of the elastomer chamber. In addition, the chamber can be pre-stretched by a freely selectable amount so as to compensate for sagging of the chamber bottom. The computer program allows, at any arbitrary time, the set drive control program to be interrupted, stopped and continued at any arbitrary time, so that the drive can be disconnected from the mains and moved to a different location, for example when the site of the experiment changes from a CO₂ incubator to a microscope. Because the computer program can also run on removable media, it can likewise be transferred from one PC to another PC. The experiment, which in the example was interrupted, can thus be continued on a different PC. Moreover, the computer program has an interface for transmitting signals to other programs that are running, for example for actuating cameras, so as to take pictures during an experiment. All program settings can also be logged as TXT files. The drive parameters selected by the user for experiments can be arbitrarily stored and retrieved as setup settings.

The entire cell stretch system is placed on a clamping frame, which can be attached directly to a cell microscope. Depending on the objective, time-resolved examinations are thus also possible. The compact design of the entire system also allows it to be uses in CO₂ incubators.

Advantageous uses of the device according to the invention include stretch experiments for cells, and more specifically, uniaxial stretch experiments.

For this purpose, cells are seeded on, and adhere to, the bottom. The advantageous use of the device according to the invention includes calibrating and varying the transverse contraction of the elastomer, depending on the applied stretch, by way of the microstructure.

The elastomer is predestined for use in uniaxial stretch experiments using a cell stretcher, which can produce tension exclusively in the X-direction of the elastomer. Because the wall thickness of the device according to the invention can be designed differently in the different edge regions, bidirectional cell stretchers are unnecessary, because the transverse contraction of the elastomer chamber can be freely adjusted by varying the edge thickness of the elastomer chamber.

According to a particularly advantageous use, the edge region of the elastomer is reinforced in the direction of tension of the cell stretcher with non-elastomer-containing materials.

Further advantages of the device are that the angle of the orientation of cells can be precisely determined by way of the microstructures, the orientation angles of the cells can be freely determined in subsequent experiments as a function of the transverse contraction, and the transverse contraction can be adapted in situ to the conditions of the cell group.

Moreover, the chamber bottom exhibits, in all locations, similar, and notably detectable, behavior during the stretch.

The invention will be described in more detail hereafter based on exemplary embodiments and the accompanying drawings. The stated dimensions and materials shall be understood to be by way of example, and to have no limiting effect.

In the drawings:

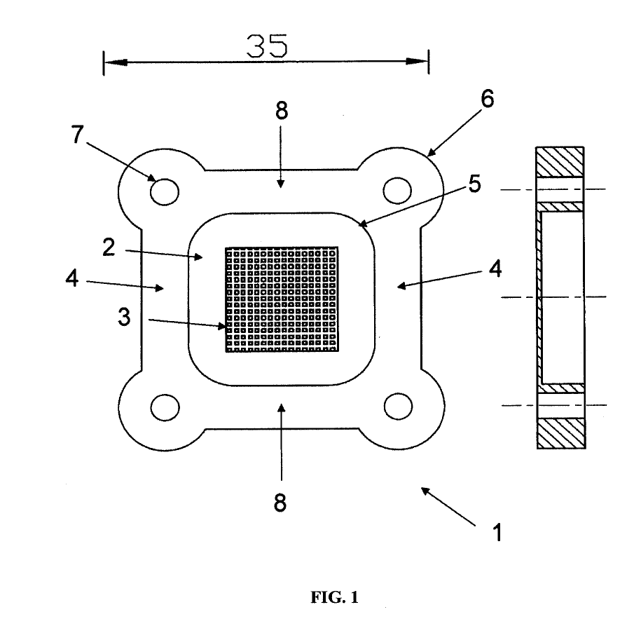

FIG. 1: is an elastomer-cell chamber system;

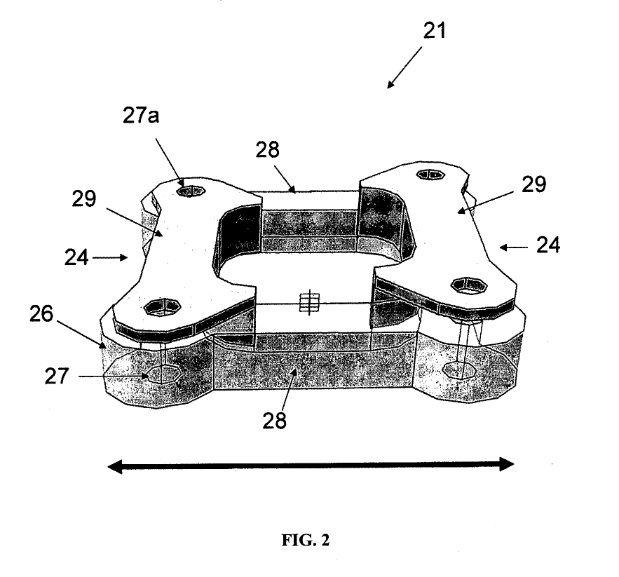

FIG. 2: is an elastomer-cell chamber system comprising two clamp-like reinforcements; and

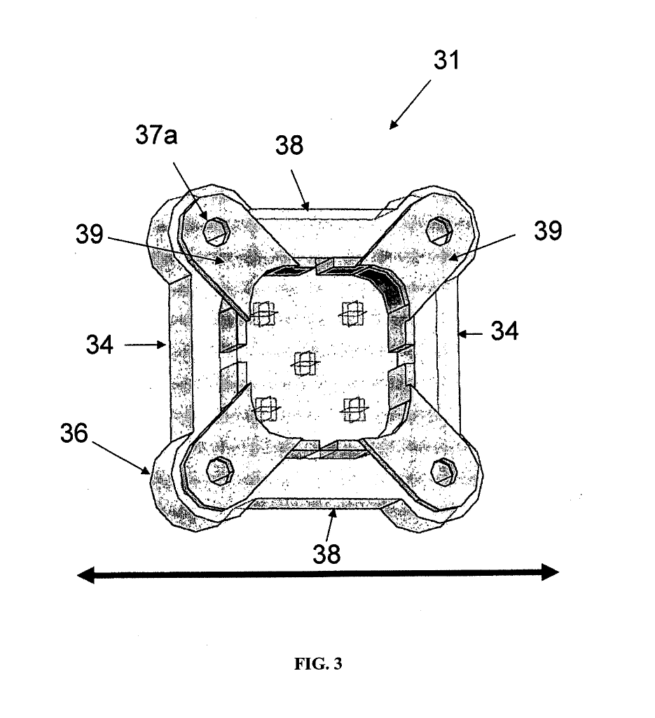

FIG. 3: is an elastomer-cell chamber system comprising four clamp-like reinforcements.

### FIRST EXEMPLARY EMBODIMENT

FIG. 1 shows schematic top and cross-sectional views of the elastomer according to the invention, which is used as a structured cell chamber system for stretch experiments for cells.

In terms of chemistry, the elastomer 1 comprises a vinyl-terminated polydimethylsiloxane as the base substance. The cross-linking agent used was a methylhydrosiloxane-dimethylsiloxane copolymer. During production, first the base substance and the cross-linking agent were mixed, degassed, and then poured into a negative mold for the cell chamber. The platinum catalyst was admixed to the base substance, and produced the cross-linking at 60° C. over night. After curing, the elasticity of the elastomer is approximately 50 kPa.

The cell chamber system 1 has a quadratic shape, as a first approximation. The side length is 35 millimeters. Each of the four corners 6 is reinforced. This means that the edge regions 8 and 4 are each only 5 millimeters thick, while the corners 6 have semi-circular, ear-shaped round reinforcements which protrude over the remaining outer edge 4, 8 and have a diameter of 10 millimeters. The corners are rounded both outwardly and inwardly.

This measure advantageously protects the corners in the stretch experiment from the tensile forces exerted via the fastening pins (not shown) of the stretcher in the openings 7 so that they do not tear easily or become damaged.

A opening 7 penetrating the elastomer material is provided in each of the corners 6. The opening 7 has a diameter of 3 millimeters and is arranged centrally in each corner 6.

The openings 7 are produced directly during the cross-linking of the cell chamber 1. The base substance and the copolymer are poured in a negative mold containing pins having a diameter similar to that of the openings 6, and are cured. In the stretch experiment, the chamber 1, with the openings 7 thereof, is placed on pins which have diameters corresponding to those during the cross-linking in the production method. Thus, in the experiment, the openings 7 have a perfect positive fit and seat with respect to the cell stretcher and the fastening pins thereof (not shown). This advantageously brings about an exact transfer of the stretch to the chamber bottom 3. In addition, risk of the elastomer tearing is minimized due to the rounded reinforcements of the corners 6.

Moreover, the four corners 6 are also rounded at the inner edge regions 5. This measure alone advantageously causes an exact transfer of the applied stretch to the chamber bottom.

Additionally, the risk of the elastomer 1 tearing is further minimized. The thickness of the elastomer in the edge regions 4, 6, 8 is a uniform 5 millimeters. In the opposing edge regions 4 or 8, the thickness (or depth) is a uniform 5 millimeters, while it is 10 millimeters in the four corners 6.

In contrast to the edge regions 4, 6, 8, the bottom region 2, 3 has a considerably smaller thickness, typically of 0.1 to 0.5 millimeters. The bottom comprises the structured central region 3 and a non-structured region 2 arranged between this central region and the edge regions 4, 6, 8. The central region 3 has regular structures in the form of elevations and depressions. The elevations and depressions, by way of example, have a diameter of 2 μm and uniform distance of 1.5 μm with respect to each other. The depth of the structures typically ranges between 50 and 500 nm, depending on the elasticity of the chamber. In the stretch experiment for the cells seeded on the structure (not shown), this structure serves as a ruler or gauge, which is used to check how the applied amplitude of the cell stretcher is transmitted to the chamber bottom, and thus to the cells. Therefore, the structure 3 in the central region of the bottom, for the first time, allows the difference between the stretch that is applied to the elastomer chamber and the stretch that is actually received at the cells to be exactly determined.

### SECOND EXEMPLARY EMBODIMENT

FIG. 2 shows a cell chamber 21 which is identical to FIG. 1, and again has a central opening 27 in each of the four corners 26. Contrary to FIG. 1, the elastomer, which is designed as the chamber 21, is equipped with a total of two clamp-like reinforcements 29 on the opposing edge regions 24 and on the corners 26. The reinforcements 29 are placed with precise and positive fit on the edge regions and the openings. The clamps 29 advantageously provide additional protection from damage to the edges by the developing tensile forces, which are represented by the thick arrow. In the present example, the clamps 29 are made of anodized aluminum.

In FIG. 2, the direction of tension in the stretch experiment is indicated by the thick arrow in the X-direction. If the opposing edge regions 24 are thicker, which is to say designed thicker in the X-direction, than the edge regions 28 arranged perpendicularly thereto, for example twice as thick, the compression in the Y-direction is increased, and thus the transverse contraction is increased. This allows the ratio of the stretch of the measuring chamber in the X-direction to the compression thereof in the Y-direction, transverse to the direction of tension, to be set by varying the thickness of the edge regions 24:28. As a result, a variety of additional exemplary embodiments are conceivable.

### THIRD EXEMPLARY EMBODIMENT

FIG. 3 shows a substantially identical cell chamber 31, which again has a central opening in each of the four corners 36. As differs from FIGS. 1 and 2, the elastomer 31 is provided with a total of four clamp-like reinforcements 39 directly on the corners 36 alone. The reinforcements 39 are placed with precise and positive fit on the corners 36 and the openings. The clamps 39 advantageously provide additional protection from damage to the edges by the developing tensile forces, which are represented by the thick arrow in the X-direction. Moreover, and in addition to the action of the clamps 29 in FIG. 2, the clamps 39 prevent the opposing edge regions 38 from deflecting. The edge reinforcements in FIG. 3 can thus be used in unidirectional stretchers to minimize the transverse contraction.

Edge reinforcements such as in FIG. 3 can advantageously also be used in bidirectional stretch systems.

The clamps 39 are made of anodized aluminum. The thickness and dimensions depend on the chamber design that is used. In the selected exemplary embodiment, the material thickness of the clamps 39 is typically 0.5 to 1.0 millimeters. The shaping follows the dimensions of the elastomer chamber; at a chamber thickness or height of 5 millimeters, the engagement depth of the clamp is typically 4.5 millimeters.

The clamps in FIGS. 2 and 3 particularly advantageously amplify the transmission of the stretch to the thin elastomer bottom. They are arranged positively in the elastomer and thereby prevent the deflection of the edge regions 24, 34 arranged in the direction of tension (thick arrow).

### FURTHER EXEMPLARY EMBODIMENTS

Further exemplary embodiments relate to measurement chambers, as shown in FIG. 2 and FIG. 3. These exemplary embodiments are produced without the side walls 28, 38 and are used in cell stretchers. Maximum transverse contraction is achieved when the bottom of the elastomer chamber is freely suspended (no edge reinforcement 28, 38).

1. A device for examining cells, comprising an elastomer for accommodating the cells, characterized in that the elastomer comprises an inwardly arranged bottom and a thicker edge region, and regular microstructures are arranged in the bottom.

2.-14. (canceled)

15. A device for examining cells, comprising an elastomer which has an inwardly arranged bottom having regular microstructures for accommodating the cells and an edge region which is thicker as compared to the bottom and has at least one opening penetrating the elastomer for fastening to a cell stretch system, the microstructures allowing a reorientation of the cells adhering to the microstructures based on a force which is caused by a stretch of the elastomer by the cell stretch system and which is exerted on the cells.

16. The device according to claim 15, wherein the transverse contraction of the elastomer and the force acting on the cells can be determined by way of the microstructures based on the stretch by the cell stretch system.

17. The device according to claim 15, wherein the elastomer has a tub shape.

18. The device according to claim 17, wherein the elastomer comprises an edge region having rounded corners in which the opening is, or openings are, arranged.

19. The device according to claim 17, wherein the elastomer has a shape in which the thicknesses of the walls of opposing edge regions are identical and in which non-opposing edge regions have differing wall thicknesses.

20. The device according claim 19, wherein the edge region of the elastomer is interrupted or left entirely open in some regions.

21. A device according to claim 15, wherein the wall thickness of the corners of the edge region of the elastomer is greater than the wall thickness in the remaining edge region.

22. A device according to claim 15, wherein a portion of the edge region of the elastomer is reinforced with non-elastomer-containing materials.

23. The device according to claim 22, comprising clamp-like reinforcements that have a positive fit relative to the elastomer.

24. Use of a device according to claim 15, in stretch experiments for cells adhering to the bottom, wherein the reorientation of the cells and/or the transverse contraction of the elastomer, which depends on the stretch applied by a cell stretch system, are measured by way of the microstructure.

25. Use according to claim 24, wherein the transverse contraction of the elastomer chamber is varied by varying the edge thickness of the elastomer chamber.

26. Use according to claim 24, wherein the edge region of the elastomer is reinforced in the direction of tension of the cell stretch system with non-elastomer-containing materials.

27. A method for producing a device according to claim 15, wherein the opening is, or the openings are, produced during the cross-linking of the elastomer.
